(19) United States
(12) Patent Application Publication
(10) Pub. No.: US 2009/0318910 A1
(43) Pub. Date: Dec. 24, 2009

(54) STRUCTURE OF MICRO LASER BEAM IRRADIATION FOR FRACTIONAL MICRO ABLATION AND METHOD OF IRRADIATION

(75) Inventors: Hae Lyung Hwang, Gyeonggi-do (KR); Chi Dae Park, Gyeonggi-do (KR); Sung Huan Gong, Seoul (KR)

Correspondence Address: PEPPER HAMILTON LLP, ONE MELLON CENTER, 50TH FLOOR, 500 GRANT STREET, PITTSBURGH, PA, 15219, US

(73) Assignee: LUTRONIC CORPORATION, Gyeonggi-do (KR)

(21) Appl. No.: 12/294,379

(22) PCT Filed: Apr. 26, 2006
(86) PCT No.: PCT/KR06/01578

§ 371 Date: Dec. 8, 2008

(30) Foreign Application Priority Data
Mar. 27, 2006 (KR) 10-2006-0027292

(51) Int. Cl. A61B 18/20

(52) U.S. Cl. 606/13

(57) ABSTRACT

The present invention relates to a micro laser beam irradiation structure and method for micro fractional ablation. A micro laser beam irradiation structure for micro fractional ablation comprises a scanner for enabling laser beams received from a light source for generating the laser beams to be irradiated in predetermined directions; and an interface unit for representing an accumulated density of the laser beams irradiated from the scanner. A micro laser beam irradiation method for micro fractional ablation comprises randomly irradiating, by a scanner, micro laser beams introduced into a handpiece, within the coverage of the area of a tip of the handpiece. According to the present invention, heat generated on the skin through random laser beam irradiation in a micro fractional ablation treatment using a laser can be prevented from being accumulated, and at the same time, damage to the skin due to heat accumulation can be minimized by accurately representing a total amount of laser treatment.

### TECHNICAL FIELD

The present invention relates to a micro laser beam irradiation method for micro fractional ablation, and more particularly, to a micro laser beam irradiation method for micro factional ablation, wherein heat generated on the skin through random laser beam irradiation in a micro fractional ablation treatment using a laser can be prevented from being accumulated, and at the same time, damage to the skin due to heat accumulation can be minimized by accurately representing a total amount of laser treatment.

### BACKGROUND ART

As shown in FIG. 1, a conventional laser for micro fractional ablation sequentially irradiates laser beams as a handpiece moves. In this case, since the laser beams are irradiated onto a part of the skin and then onto a next part close to the part on which the laser beams have been irradiated, energy absorbed by skin tissue is accumulated so that the temperature of the skin tissue increases. Further, since the laser beams are continuously irradiated with a high density onto a small area on the skin without sufficient cooling time, excessive heat may be accumulated on the skin tissue. As a result, there is a problem in that the skin tissue may be damaged due to side effects such as pigmentation or edema.

Further, the laser for micro factional ablation should secure an appropriate laser beam density in order to obtain clinical effects, but should not exceed a predetermined laser beam density in order to avoid its side effects. Therefore, it is very important to accurately control an accumulated laser beam density. Generally, the laser beams are irradiated with a density of 700 to 2000 beams/cm².

However, in the prior art, there are problems in that the skin is damaged due to excessive laser beam irradiation since a user cannot know how many laser beams have been irradiated to the skin, and that clinical effects are deteriorated due to an insufficient total amount of laser beam irradiation.

### DISCLOSURE OF INVENTION

### Technical Problem

Accordingly, the present invention is conceived to solve the aforementioned problems. An object of the present invention is to provide a micro laser beam irradiation method for micro factional ablation, wherein heat generated on the skin through random laser beam irradiation in a micro fractional ablation treatment using a laser can be prevented from being accumulated, and at the same time, damage to the skin due to heat accumulation can be minimized by accurately representing a total amount of laser treatment.

### Technical Solution

To achieve the object of the present invention, a micro laser beam irradiation structure for micro fractional ablation according to the present invention comprises a scanner for enabling laser beams received from a light source for generating the laser beams to be irradiated in predetermined directions; and an interface unit for representing an accumulated density of the laser beams irradiated from the scanner.

In the micro laser beam irradiation structure, a tip provided at a lower portion of the scanner may have a width of 3 mm to 80 mm.

A micro laser beam irradiation method for micro factional ablation according to the present invention comprises randomly irradiating, by a scanner, micro laser beams introduced into a handpiece, within the coverage of the area of a tip of the handpiece. The handpiece may represent an accumulated amount of the irradiated laser beams per unit area.

### Advantageous Effects

The present invention constructed as above has the following advantages.

According to the present invention, heat generated on the skin through random laser beam irradiation in a micro fractional ablation treatment using a laser can be prevented from being accumulated, and at the same time, damage to the skin due to heat accumulation can be minimized by accurately representing a total amount of laser treatment.

### BRIEF DESCRIPTION OF THE DRAWINGS

FIG. 1 is a schematic view showing a state where micro laser beams are irradiated in a prior art.

FIG. 2 is a schematic view showing a micro laser beam irradiation structure according to the present invention.

FIG. 3 is a schematic view showing an irradiation distribution of micro laser beams in accordance with the present invention.

FIGS. 4a and 4b are schematic view showing laser beam irradiation types in the irradiation of the micro laser beams shown in FIG. 3.

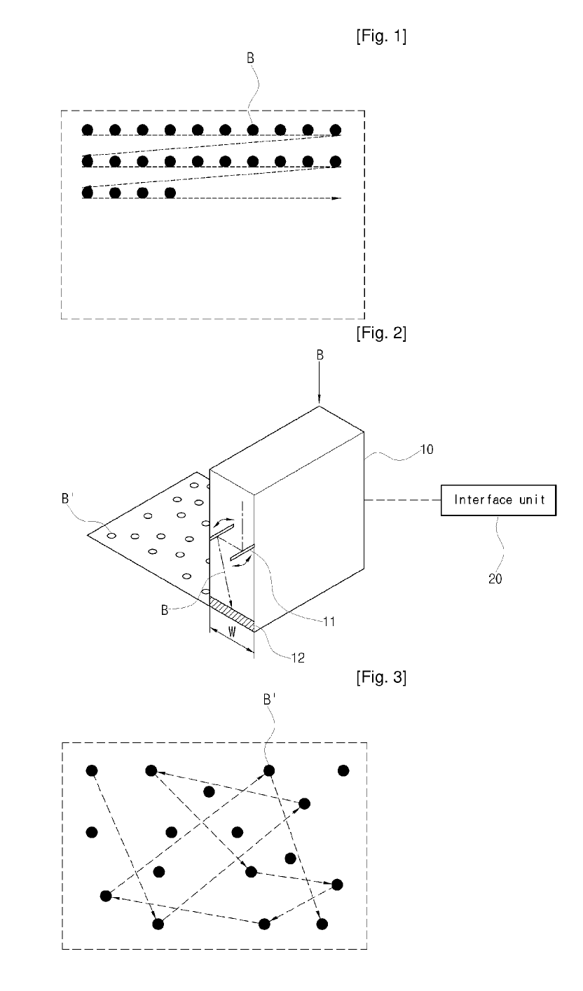

### BEST MODE FOR CARRYING OUT THE INVENTION

Hereinafter, a preferred embodiment of the present invention will be described in detail with reference to the accompanying drawings.

As shown in FIG. 2, a handpiece 10 includes a condensing unit (not shown) for condensing laser beams emitted from a light source for generating the laser beams, a scanner 11 for non-uniformly irradiating the laser beams received from the condensing unit, and an interface unit 20 for representing an accumulated density of the laser beams irradiated by the scanner.

Here, the condensing unit applies the laser beams each of which has a size of 50 μm to 200 μm, wherein the applied laser beams B are irradiated in predetermined directions by means of changes in reflection angles of mirrors included in the scanner 11. At this time, random control of the changes in the reflection angles of the mirrors causes laser beams B′ irradiated onto the skin to be irregularly distributed on parts of the skin, thereby preventing the skin from being thermally damaged due to the irradiation of the laser beams and simultaneously facilitating quick heat dissipation.

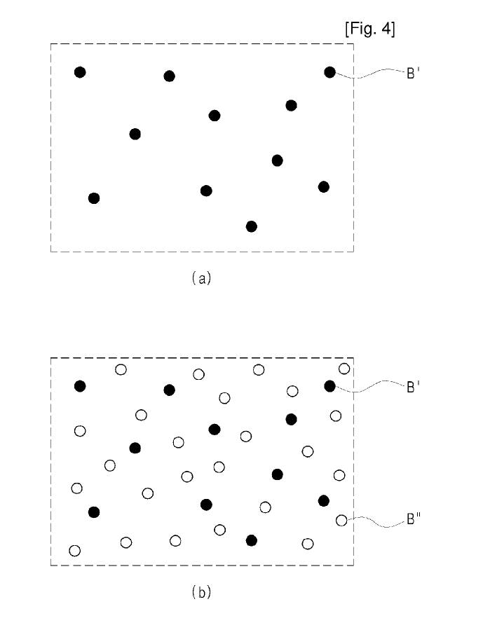

For example, a tip 12 provided at a lower portion of the handpiece 10 has a width W of 3 mm to 80 mm, so that the laser beams can be randomly irradiated within the coverage of the tip 12. As shown in FIG. 3, an interval between successively irradiated laser beams is caused to be increased, so that heat can be dissipated to the surroundings from parts to which the laser beams B′ have already been irradiated as shown in FIGS. 4a and 4b. Accordingly, even if the next laser beams B″ are irradiated, thermal accumulation may not be generated, thereby preventing thermal damage to the skin.

Further, in the laser beam irradiation for micro fractional ablation, the density of laser beams that can be irradiated at a time is limited to about 500 beams/cm² depending on the quantity of energy. If laser beams with a laser beam density of 500 beams/cm² or more are irradiated on the skin tissue at a time, the skin may be occasionally damaged depending on the quantity of energy. Thus, it is preferred that laser beams be repeatedly irradiated several times on an identical part of the skin. When the laser beams are repeatedly irradiated on the identical part of the skin as described above, it is necessary to accurately inform a user how large the density of the irradiated laser beams becomes. At this time, the area of a part of the skin to be treated is input into a control unit (not shown) before the treatment is performed. The number of laser beams to be irradiated on the area of the part of the skin is then calculated. The density of the laser beams which have been irradiated up to date is represented in real time to a user by using the interface unit 20. In order to input the area of a part of the skin to be treated, any one method may be selected among a method of marking a grid pattern on the skin, counting the number of related scales and inputting the counted number; a method of calculating an area using a tape measure or the like and inputting the calculated area; a method of covering the part of the skin with a transparent mask with scales printed thereon, counting the number of related scales and inputting the counted number; a method of selecting a standard size of a face or the like; and the like.

Hereinafter, the micro laser beam irradiation method for micro fractional ablation according to the present invention will be described.

In the laser beam irradiation method, laser beams introduced into the handpiece are controlled to be irregularly reflected by the scanner so that they can be randomly irradiated. It is preferred that the accumulated amount of the laser beams irradiated per unit area be represented. In the micro fractional ablation treatment, each of the laser beams is caused to have a very small size, e.g., 50 μm to 200 μm, and then be irradiated on the skin. At this time, the micro laser beam has a penetration depth of up to 4 mm depending on its wavelength. Further, in this micro fractional ablation treatment, the micro laser beam is not irradiated throughout the entire surface of the skin but is discretely irradiated on the skin. Therefore, a large part of the surface of the skin is not ablated as a whole, but the ablation is made to limited minute parts of the surface of the skin. When the number of minute parts to be ablated is very large, it is possible to obtain effects that 10 to 20% of a total area is ablated at a time.

Accordingly, laser beams to be irradiated are formed to have a small size and the laser beams are randomly irradiated using the scanner within a range in which the laser beams can be irradiated by the handpiece, thereby preventing thermal damage to the skin tissue due to the laser beams irradiated on the skin tissue.

Further, the number of the irradiated laser beams is calculated so that a user can always confirm the accumulated number of the irradiated laser beams per unit area, thereby treating the skin tissue while minimizing damage thereto. Herein, in order to know the accumulated number of the irradiated laser beams per unit area, the area of a part of the skin to be treated is first calculated or measured and then input into the control unit which in turn calculates the total number of laser beams to be irradiated on the input area. At this time, the laser beams are irradiated within the coverage of the cross-sectional area of the handpiece tip. In order to input the area of a part of the skin to be treated, any one method may be selected among a method of marking a grid pattern on the skin, counting the number of related scales and inputting the counted number; a method of calculating an area using a tape measure or the like and inputting the calculated area; a method of covering the part of the skin with a transparent mask with scales printed thereon, counting the number of related scales and inputting the counted number; a method of selecting a standard size of a face or the like; and the like.

By irradiating laser beams using such a method as described above, the skin tissue to be treated can be treated with minimized damage thereto.

The present invention is not limited to the embodiments described above, and those skilled in the art can make various modifications and changes thereto. The modifications and changes fall within the spirit and scope of the present invention defined by the appended claims.

1. A micro laser beam irradiation structure for micro fractional ablation, the structure comprising:
a scanner for enabling laser beams received from a light source for generating the laser beams to be irradiated in predetermined directions; and
an interface unit for representing an accumulated density of the laser beams irradiated from the scanner.

2. The micro laser beam irradiation structure as claimed in claim 1, wherein a tip provided at a lower portion of the scanner has a width of 3 mm to 80 mm.

3. A micro laser beam irradiation method for micro factional ablation, comprising: randomly irradiating, by a scanner, micro laser beams introduced into a handpiece, within the coverage of the area of a tip of the handpiece.

4. The micro laser beam irradiation method as claimed in claim 3, wherein the handpiece represents an accumulated amount of the irradiated laser beams per unit area.
